SEQUENCE LISTING (3 sequences)

SEQ ID NO: 1 - DNA, 80 bp, Artificial
  Antisense oligonucleotide
tggagttgaaaaagcttgactggcgtcattcaggagctggatggcgtgggacatgtgcaaccaggactctgagtctgtat

SEQ ID NO: 2 - DNA, 80 bp, Artificial
  Antisense oligonucleotide
tgctctgtgtcactgtggattggagttgaaaaagcttgactggcgtcattcaggagctggatggcgtgggacatgtgcaa

SEQ ID NO: 3 - DNA, 80 bp, Artificial
  Antisense oligonucleotide
tggcgtcattcaggagctggatggcgtgggacatgtgcaaccaggactctgagtctgtatggagtgacatcgagtgtgct

(19) United States
(12) Patent Application Publication
(10) Pub. No.: US 2009/0029933 A1
(43) Pub. Date: Jan. 29, 2009

(54) INHIBITOR OF PEROXISOME PROLIFERATOR-ACTIVATED RECEPTOR ALPHA COACTIVATOR 1

(76) Inventors: Licio Augusto Velloso, Campinas (BR); Claudio Teodoro de Souza, Campinas (BR)

Correspondence Address: LADAS & PARRY LLP, 224 SOUTH MICHIGAN AVENUE, SUITE 1600, CHICAGO, IL, 60604, US

(21) Appl. No.: 11/909,323

(22) PCT Filed: Mar. 20, 2006
(86) PCT No.: PCT/BR2006/000055

§ 371 Date: Jun. 23, 2008

(30) Foreign Application Priority Data
Mar. 23, 2005 (BR) PI 0500959-6

(51) Int. Cl. A61K 31/711; C07H 21/04; A61K 48/00; C12N 15/63; A61P 3/10

(52) U.S. Cl. 514/44; 536/23.1; 435/320.1

(57) ABSTRACT

The present invention refers to the use of an antisense DNA oligonucleotide for the messenger RNA of the PGC-1α protein, useful as drug for the treatment of diabetes mellitus, insulin resistance and metabolic syndrome. More specifically, the present invention deals with a compound used as drug, through enteral or parenteral route, preferably, with the property of inhibiting the protein expression peroxisome proliferator-activated receptor alpha Coactivator 1 (PGC-1α) leading to the reduction of the blood glucose levels. It deals, therefore, with a pharmacological compound that promotes, in diabetic and insulin-resistant individuals, improvement of the glucose serum levels, increase of the plasmatic insulin concentration and reduction of insulin resistance. The present invention presents a more effective control of the glucose levels and acts beneficially on other complications associated to the Diabetes and obesity conditions, according to tests performed in animal models. In this manner, the principal advantage of the present invention over others alike already existing in the market is the effectiveness that controls blood glucose levels and the fact of acting beneficially on other complications that accompany the disease.

### FIELD OF THE INVENTION

The present invention deals with the use of an oligonucleotide as a drug for the treatment of diabetes mellitus, insulin resistance and metabolic syndrome.

More specifically, the present invention deals with a compound used as a drug, by enteral or parenteral route, with the property of inhibiting the expression of the protein peroxisome proliferator-activated receptor alpha Coactivator 1 (PGC-1α), leading to the reduction of the blood glucose levels. It deals therefore with a pharmacological compound that promotes, in diabetic individuals and those resistant to insulin, improvement of the glucose serum levels, increase of plasmatic insulin concentration and reduction of the resistance to insulin. The present invention is of great social interest, and on a commercial scope, it is of great interest to the pharmaceutical industry.

### BASIS OF THE INVENTION

During the last decades a progressive increase of the prevalence of obesity and type 2 diabetes mellitus was observed in several regions of the world (Kopelman P G 2000 Obesity as a medical problem. Nature 404:635-43; Flier J S 2004 Obesity wars: molecular progress confronts an expanding epidemic. Cell 116:337-50; Stein C J, Colditz G A 2004 The epidemic of obesity. J Clin Endocrinol Metab 89:2522-5). Modifications of food intake patterns and sedentarism acting on favorable genetic backgrounds are indicated as the most important causal factors for these diseases. Type 2 diabetes mellitus and obesity are closely associated. A 1.0 kg/m² increase in the body mass index can double the relative risk for the development of diabetes (Kopelman P G 2000 Obesity as a medical problem. Nature 404:635-43). In an epidemiological evaluation performed in Brazil in the year 2000 it was concluded that 9% of the population presented diabetes mellitus and 15% were obese (Kopelman P G 2000 Obesity as a medical problem. Nature 404:635-43). The same study presented projections for the year 2020 concluding that, in case no important modifications occur in the treatment modalities of these diseases, the prevalence of diabetes should reach 15% and that of obesity should exceed 25% (Kopelman P G 2000 Obesity as a medical problem. Nature 404:635-43).

Body weight maintenance depends on a complex equilibrium between ingestion of calories and energy consumption. Positive energetic balance leads to a progressive storage of the caloric surplus, in the form of triglycerols in the adipose tissue, which, when maintained for an extended period of time, will result in the development of obesity (Schwartz M W, Kahn S E 1999 Insulin resistance and obesity. Nature 402:860-1). While acquisition of energy depends exclusively on the ingested food, energy consumption is a result of a series of factors that, when summed up, will contribute to the total energy consumption of a determined individual. (Schwartz M W, Kahn S E 1999 Insulin resistance and obesity. Nature 402:860-1; Schwartz M W, Woods S C, Porte D, Jr., Seeley R I, Baskin D G 2000 Central nervous system control of food intake. Nature 404:661-71). These factors include physical activity and the two forms of thermogenesis, obligatory and adaptive. (Schwartz M W, Kahn S E 1999 Insulin resistance and obesity. Nature 402:860-1; Schwartz M W, Woods S C, Porte D, Jr., Seeley R J, Baskin D G 2000 Central nervous system control of food intake. Nature 404:661-71). The therapeutics of obesity centered on the increase of the physical activity does not result in satisfactory weight loss, which suggests that sedentarism, per se, must play a minor role in the pathogenesis of obesity and consequently of diabetes mellitus. On the other hand, defects in thermogenesis are regarded as important factors for the development of these diseases (Schwartz M W, Kahn S E 1999 Insulin resistance and obesity. Nature 402:860-1; Schwartz M W, Woods S C, Porte D, Jr., Seeley R I, Baskin D G 2000 Central nervous system control of food intake. Nature 404:661-71).

The molecular mechanisms involved in heat generation are diverse. There are metabolic cycles that promote ATP consumption with a subsequent release of heat, like for example, the glycolytic and gluconeogenic cycle, or even the Na+, K+ ATPase activity. Yet, heat can be released through ATP hydrolysis as what happens during shivering thermogenesis. However, in parallel to such cellular mechanisms, interference in the electron transport chain in the mitochondria has been characterized as one of the most potent heat production and energy consumption mechanisms. In accordance with Mitchell's chemiosmotic theory, electron transport through the cytochrome chain of the inner mitochondrial membrane generates a proton gradient that activates the enzyme ATP synthase resulting in synthesis of ATP. The term mitochondrial coupling precisely refers to the capacity of the mitochondria in adapting the rhythm of oxidation to energy demand. From the functional point of view, the presence of ADP results in an increase of the respiratory rhythm (state 3), to a pace that, in the absence of ADP (state 4), the failure of this rhythm occurs. The relation between state 3 and state 4 (state 3/state 4) reveals the degree of mitochondrial coupling. Therefore, mitochondrial uncoupling results from any mechanism that is capable of dissipating the proton gradient and, thus interfering in the state 3/state 4 relation. Such dissipation leads to heat production in detriment of the production of ATP. (Argyropoulos G, Harper M E 2002 Uncoupling proteins and thermoregulation. J Appl Physiol 92:2187-98).

Mitochondrial uncoupling proteins (UCP's) fulfill the physiological role of dissipating the proton gradient and therefore interfering in the state 3/state 4 relation. The result of the UCPs' activity is the generation of heat in detriment of the activation of ATP synthase. The first protein of this family (UCP-1) was identified, two decades ago, on brown adipose tissue, which has been initially denominated thermogenin (Maia I G, Benedetti C E, Leite A, Turcinelli S R, Vercesi A E, Arruda P 1998 AtPUMP: an Arabidopsis gene encoding a plant uncoupling mitochondrial protein. FEBS Lett 429:403-6; Bukowiecki L J 1984 Mechanisms of stimulus-calorigenesis coupling in brown adipose tissue. Can J Biochem Cell Biol 62:623-30). It is characterized as a 32 kDa protein that is activated by adrenergic stimuli, which promotes the activation of cyclic AMP resulting the conversion of triglycerols into free fatty acids, these in turn activate the UCP-1 leading to the uncoupling of the mitochondrial respiration. UCP-1 can also be regulated through mechanisms that control the transcription of its gene, where the sympathetic tonus is also an important inductor of this phenomenon (Palou A, Pico C, Bonet M L, Oliver P 1998 The uncoupling protein, thermogenin. Int J Biochem Cell Biol 30:7-11).

In 1997, two other proteins pertaining to the UCP's family were identified, which were denominated UCP-2 and UCP-3. The first is expressed in several tissues and the second predominantly in skeletal muscular tissue. Finally, in more recent years, two new proteins pertaining to the same family, but with degrees of homology lower than those of the first ones, were identified, which are called UCP-4 and UCP-5 (Argyropoulos G, Harper M E 2002 Uncoupling proteins and thermoregulation. J Appl Physiol 92:2187-98).

Different experimental evidences suggest the participation of the UCP's in uncoupling and therefore in thermogenesis control. As previously said, UCP-1 present in brown adipose tissue is controlled by sympathetic stimuli that, through the induction of molecular mechanisms, control the production of free fatty acids and modulate the activity of the UCP, besides this, the same neural stimulus activates transcriptional programs that increase the UCP-1 protein expression (Argyropoulos G, Harper M E 2002 Uncoupling proteins and thermoregulation. J Appl Physiol 92:2187-98). In the same context, the UCP-2 ectopic expression or the UCP-3 transgenic hyperexpression lead to the increase of thermogenesis through mitochondrial uncoupling-dependent mechanism. Therefore, it remains evident that the UCP family proteins play a central role in the mechanisms of energy consumption and heat production (Chan C B, MacDonald P E, Saleh M C, Johns D C, Marban E, Wheeler M B 1999 Overexpression of uncoupling protein 2 inhibits glucose-stimulated insulin secretion from rat islets. Diabetes 48:1482-6; Chan C B, De Leo D, Joseph J W, McQuaid T S, Ha X F, Xu F, Tsushima R G, Pennefather P S, Salapatek A M, Wheeler M B 2001 Increased uncoupling protein-2 levels in beta-cells are associated with impaired glucose-stimulated insulin secretion: mechanism of action. Diabetes 50:1302-10).

Due to their important role in cellular energy flux control, the UCP family proteins soon became the focus of research that aimed at developing pharmacological mechanisms that would induce their activity. Such compounds, if developed successfully, would have potential use in the therapeutics of obesity and similar diseases.

The first experimental approaches aimed at evaluating the functional regulation effects of the UCP proteins, came through the breeding of transgenic and knockouts animals. The disarrangement of the UCP-1 gene, leading to the total absence of its expression, did not promote important changes in body weight or food intake but led to an exaggerated sensitivity to cold exposure (Melnyk A, Himms-Hagen J 1998 Temperature-dependent feeding: lack of role for leptin and defect in brown adipose tissue-ablated obese mice. Am J Physiol 274:R1131-5). On the other hand, the transgenic induction of the UCP-1 ectopic expression on skeletal muscle, turned the animals resistant to diet induced obesity (Argyropoulos G, Harper M E 2002 Uncoupling proteins and thermoregulation. J Appl Physiol 92:2187-98). Besides this, blood glucose and insulin levels became lower, suggesting greater sensitivity to the pancreatic hormone. Finally, the cholesterol levels were also lower in these mice. In addition, animals with gene ablation of the UCP-2 expression did not become obese, however, different from the UCP-1 knockout animals, these were not sensitive to cold exposure. On the other hand, upon chasing by an infectious condition, the UCP-2 knockout mice presented greater production of free radicals, being in this manner more apt to fight the infection. The ablation of the UCP-2 expression in ob/ob mice, which develops obesity and diabetes mellitus due to a recessive monogenic defect that suppresses leptin hormone production, led to an increase in insulin production and improved the glycemic levels. (Chan C B, MacDonald P E, Saleh M C, Johns D C, Marban E, Wheeler M B 1999 Overexpression of uncoupling protein 2 inhibits glucose-stimulated insulin secretion from rat islets. Diabetes 48:1482-6; Chan C B, De Leo D, Joseph J W, McQuaid T S, Ha X F, Xu F, Tsushima R G, Pennefather P S, Salapatek A M, Wheeler M B 2001 Increased uncoupling protein-2 levels in beta-cells are associated with impaired glucose-stimulated insulin secretion: mechanism of action. Diabetes 50:1302-10; Chan C B, Saleh M C, Koshkin V, Wheeler M B 2004 Uncoupling protein 2 and islet function. Diabetes 53 Suppl 1:S136-42). Finally, UCP-3 knockout animals did not become obese and did not present defective thermogenesis. However, such animals produced more reactive oxygen species (Zhou M, Lin B Z, Coughlin S, Vallega G, Pilch P F 2000 UCP-3 expression in skeletal muscle: effects of exercise, hypoxia, and AMP-activated protein kinase. Am J Physiol Endocrinol Metab 279:E622-9).

Interestingly, the hyperexpression of UCP-3 produced hyperphagic, thin animals with lower adipose tissue mass and with better glucose clearance (Zhou M, Lin B Z, Coughlin S, Vallega G, Pilch P F 2000 UCP-3 expression in skeletal muscle: effects of exercise, hypoxia, and AMP-activated protein kinase. Am J Physiol Endocrinol Metab 279:E622-9).

The report that UCP-2 is the protein of the UCP family with the highest expression in pancreatic islets called the attention towards its potentiality as therapeutic target in conditions where insulin secretion is insufficient for the demand. Transgenic animals in which the UCP-2 expression in pancreatic islets is reduced present greater baseline and insulin-stimulated secretion (Chan C B, MacDonald P E, Saleh M C, Johns D C, Marban E, Wheeler M B 1999 Overexpression of uncoupling protein 2 inhibits glucose-stimulated insulin secretion from rat islets. Diabetes 48:1482-6; Chan C B, De Leo D, Joseph J W, McQuaid T S, Ha X F, Xu F, Tsushima R G, Pennefather P S, Salapatek A M, Wheeler M B 2001 Increased uncoupling protein-2 levels in beta-cells are associated with impaired glucose-stimulated insulin secretion: mechanism of action. Diabetes 50:1302-10; Chan C B, Saleh M C, Koshkin V, Wheeler M B 2004 Uncoupling protein 2 and islet function. Diabetes 53 Suppl 1:S36-42). Besides this, there is a significant improvement of the diabetes condition in diabetic obese mice than in the reduced expression of this protein.

The control of the expression of the UCP genes, including UCP-2 is poorly known, however, recent studies revealed that the protein denominated peroxisome proliferator-activated receptor alpha Coactivator 1 (PGC-1α) performs an important role in this regulation (De Souza C T, Gasparetti A L, Pereira-da-Silva M, Araujo E P, Carvalheira J B, Saad M J, Boschero A C, Carneiro E M, Velloso L A 2003 Peroxisome proliferator-activated receptor gamma coactivator-1-dependent uncoupling protein-2 expression in pancreatic islets of rats: a novel pathway for neural control of insulin secretion. Diabetologia 46:1522-31).

PGC-1α is a protein composed of 795 amino acids, initially described in brown adipose tissue and skeletal muscle, through a yeast two-hybrid system (Yoon J C, Puigserver P, Chen G, Donovan J, Wu Z, Rhee J, Adelmant G, Stafford J, Kahn C R, Granner D K, Newgard C B, Spiegelman B M 2001 Control of hepatic gluconeogenesis through the transcriptional coactivator PGC-1. Nature 413:131-8). As a gene transcription coactivator, PGC-1α, has several functional domains that allow its physical interaction with transcription factors like PPARγ, PPARα, nuclear respiration factor (NRF), CREB binding protein (CBP), hepatocyte nuclear factor alpha 4 (HNF-4α), forkhead transcription factor 1 (FOXO1), steroid receptor coactivator 1 (SRC-1), and myocyte enhancer factor 2 (MEF-2). Recent studies have related PGC-1α to the control of glucose uptake and insulin action in liver and muscle (Yoon J C, Puigserver P, Chen G, Donovan J, Wu Z, Rhee J, Adelmant G, Stafford J, Kahn C R, Granner D K, Newgard C B, Spiegelman B M 2001 Control of hepatic gluconeogenesis through the transcriptional coactivator PGC-1. Nature 413:131-8; Oliveira R L, Ueno M, de Souza C T, Pereira-da-Silva M, Gasparetti A L, Bezzera R M, Alberici L C, Vercesi A E, Saad M J, Velloso L A 2004 Cold-induced PGC-1alpha expression modulates muscle glucose uptake through an insulin receptor/Akt-independent, AMPK-dependent pathway. Am J Physiol Endocrinol Metab 287:E686-95). Besides this, two clinical studies revealed that mutations in the PGC-1α gene can be related to insulin resistance and diabetes (Ek J, Andersen G, Urhammer S A, Gaede P H, Drivsholm T, Borch-Johnsen K, Hansen T, Pedersen O 2001 Mutation analysis of peroxisome proliferator-activated receptor-gamma coactivator-1 (PGC-1) and relationships of identified amino acid polymorphisms to Type II diabetes mellitus. Diabetologia 44:2220-6; Hara K, To be K, Okada T, Kadowaki H, Akanuma Y, Ito C, Kimura S, Kadowaki T 2002 A genetic variation in the PGC-1 gene could confer insulin resistance and susceptibility to Type II diabetes. Diabetologia 45:740-3).

Recent studies reveal that in primarily insulin-resistant individuals, only a failure of the β-pancreatic cell in meeting the growing demand for insulin in the periphery should lead to the development of type 2 diabetes mellitus. Therefore, pharmacological mechanisms that lead to a continuous adjustment of insulin production in clinical situations in which there is greater demand, should be useful in the therapeutics of diabetes mellitus (Moller D E 2001 New drug targets for type 2 diabetes and the metabolic syndrome. Nature 414:821-7).

Due to the potentiality of the UCP proteins and particularly of UCP-2 as therapeutic target in metabolic diseases, particularly with respect to its participation in the control of insulin secretion it would be interesting to investigate compounds capable of controlling the UCP-2 expression and thus evaluating its effects on glucose homeostasis and insulin secretion.

Diabetes Mellitus and similar conditions comprise one of the disease groups with the highest prevalence in the world.

Therefore, having in mind that effective therapeutic methods are scarce and the consequences of inadequate control of these disease are devastating, reducing significantly the life expectancy of affected individuals, the development of new therapeutic modalities, would be important and on a commercial basis, of great interest to the pharmaceutical industry. More specifically, the development of an antisense deoxyribonucleic acid oligonucleotide for the PGC-1α messenger ribonucleic acid, an important nuclear controller of the UCP expression, would have potential use in the therapeutics of diabetes mellitus and related diseases.

### BRIEF DESCRIPTION OF THE FIGURES

The following makes reference to the figures that accompany this descriptive report, for its better understanding and illustration:

FIG. 1 illustrates the effect of a lipid-rich diet (F) in comparison with standard diet for rodents (C) over the variation of the body mass (a), the baseline glucose serum levels (b) and baseline insulin plasmatic levels (c), in mice of the SW/Uni and CBA/Uni strains. The results are presented with mean±standard error of the mean of an n=6; *p<0.05.

FIG. 2 illustrates the immunoblot (IB) analysis (IB) of the PGC-1α liver and adipose tissue (WAT) expression of SW/Uni and CBA/Uni mice fed with standard diet for rodents or lipid-rich diet. Four-week old mice were randomly selected for inclusion in the group that received standard or lipid-rich diet, from time zero and every four weeks, four animals of every group were used for the acquisition of samples of liver and adipose tissue protein extracts. Such samples were then employed in immunoblot experiments with anti-PGC-1α antibody. In all n=4 experiments. The results are presented with mean±standard error of the mean.

FIG. 3 represents the immunoblot (IB) analysis of the effect of (a) increasing doses of PGC-1□/AS on the PGC-1□ expression in liver and adipose tissue (WAT) expression of SW/Uni mice. In b, a daily dose of 1.0 nmol of PGC-1□/AS (AS) was used in comparison with animals treated only with vehicle (C) or with sense control oligonucleotides (S). The expression of PGC-1α and the actin (in liver) and vimentine (in adipose tissue) structural proteins were evaluated in this experiment. The effect of PGC-1□/AS (triangles) was even tested on the baseline glucose serum levels (c), baseline insulin plasmatic levels (d), body mass (e), and food intake (f), in comparison with the vehicle (squares) or sense control oligonucleotides (circles). The results are presented with mean±standard error of the mean, n=4 (a and b) or n=6 (c-f); *p<0.05 vs. C.

FIG. 4 represents the metabolic effects of SW/Uni treatment with PGC-1□/AS. The mice were treated with 1.0 nmol/day of PGC-1□/AS (triangles, AS), or sense control (circles, S) or vehicle (squares, C) and evaluated through glucose tolerance test (a and b), insulin tolerance test (c) or euglycemic-hyperinsulinemic clamp (d). The results are presented with mean standard error of the mean of an n=6; *p<0.05.

FIG. 5 represents the effects of the treatment of SW/Uni mice with PGC-1□/AS on the IR and Akt expression (upper blots of every graph) and on the molecular activation, measured through the determination of IR tyrosine phosphorylation or in Akt serine in liver and adipose tissue. The results are presented with mean±standard error of the mean of an n=6; *p<0.05.

### BRIEF DESCRIPTION OF THE INVENTION

The present invention refers to an antisense deoxyribonucleic acid oligonucleotide for the messenger ribonucleic acid for the PGC-1α protein. This compound possesses the property of binding itself to the corresponding sequence through the pairing of bases in accordance with the Watson and Crick model and through this mechanism inhibiting the translation of the ribonucleic acid messenger in protein. Used as a drug for the treatment of diabetes mellitus, insulin resistance and metabolic syndrome.

More specifically, this compound promotes, in diabetic and insulin-resistant individuals, improvement of the glucose serum levels, increase of the plasmatic insulin concentration and reduction of insulin resistance. The compound can be, preferably, administered orally or parenterally, in the dose of 5 to 10 nmol/kg of weight, in a single daily dose in individuals with type diabetes mellitus, insulin resistance or metabolic syndrome.

Yet, more specifically, the present invention refers to a modified deoxyribonucleic acid oligonucleotide in accordance with the sequences No 1, No 2 and No 3, used as drug for enteral or parenteral administration for the treatment of type 2 diabetes mellitus, insulin resistance and metabolic syndrome.

Sequence N^{o} 1
5′-tggagttgaa aaagcttgac tggcgtcatt caggagctgg
atggcgtggg acatgtgcaa ccaggactct gagtctgtat-3′
Sequence N^{o} 2
5′-tgctctgtgt cactgtggat tggagttgaa aaagcttgac
tggcgtcatt caggagctgg atggcgtggg acatgtgcaa-3′
Sequence N^{o} 3
5′-tggcgtcatt caggagctgg atggcgtggg acatgtgcaa
ccaggactct gagtctgtat ggagtgacat cgagtgtgct-3′

Considering that the classical therapeutic methods in use for the treatment of Diabetes Mellitus and similar diseases are scarce and do not promote the desired control in the greater part of the patients, leading to innumerous secondary complications of the Diabetes Mellitus, compromising the quality of life and increasing the mortality of the affected individuals, the present invention can be seen as a solution for such problems. More specifically, the present invention leads to a more effective control of the glucose levels and acts beneficially on other complications associated to the Diabetes and obesity conditions, according to tests performed in animal models.

In this manner, the principal advantage of the present invention on others alike already existing in the market is the effectiveness that controls blood glucose levels and the fact of acting beneficially on other complications that accompany the disease.

### DETAILED DESCRIPTION OF THE INVENTION

The present invention refers to a deoxyribonucleic acid oligonucleotide in accordance with the sequences No 1, No 2 and No 3, used as drug for enteral or parenteral administration for the treatment of type 2 diabetes mellitus, insulin resistance and metabolic syndrome.

Sequence N^{o} 1
5′-tggagttgaa aaagcttgac tggcgtcatt caggagctgg
atggcgtggg acatgtgcaa ccaggactct gagtctgtat-3′
Sequence N^{o} 2
5′-tgctctgtgt cactgtggat tggagttgaa aaagcttgac
tggcgtcatt caggagctgg atggcgtggg acatgtgcaa-3′
Sequence N^{o} 3
5′-tggcgtcatt caggagctgg atggcgtggg acatgtgcaa
ccaggactct gagtctgtat ggagtgacat cgagtgtgct-3′

The present invention includes:

### EXAMPLE 1

### Effects of the Treatment of Obese and Diabetic Mice with the Antisense Oligonucleotide PGC-1α

Characterization of the Animal Model Used:

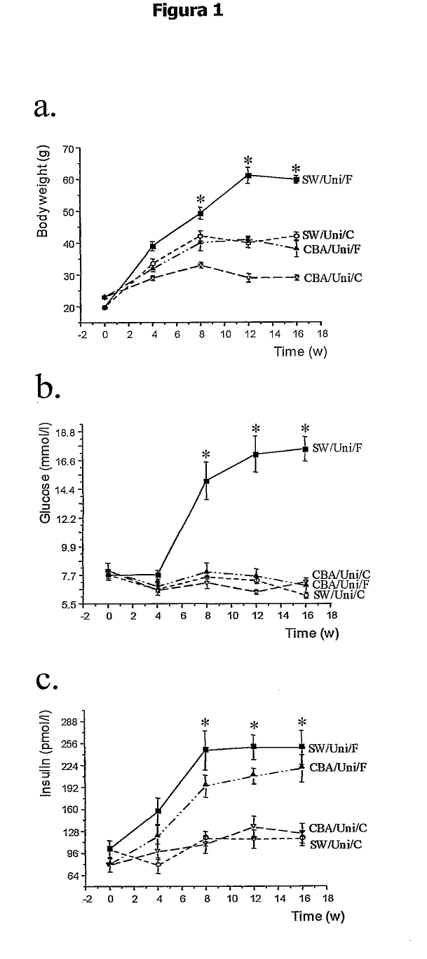

Initially, the animal model to be used in these experiments was characterized. Mice from two distinct strains were employed, however with certain genetic identity, the SW/Uni and CBA/Uni mice. Both strains are related with each other and also to the AKR mouse, previously described as possessing a predisposition for the development of diabetes and obesity when fed with lipid-rich diet (Rossmeisl M, Rim J S, Koza R A, Kozak L P 2003 Variation in type 2 diabetes-related traits in mouse strains susceptible to diet-induced obesity. Diabetes 52:1958-66). When treated with standard diet for rodents the SW/Uni and CBA/Uni mice do not develop obesity or diabetes (FIG. 1). However, when fed with lipid-rich diet the CBA/Uni mice become obese while SW/Uni become obese and diabetic, presenting the baseline glucose serum levels higher than 16.0 nmol/l (FIG. 1).

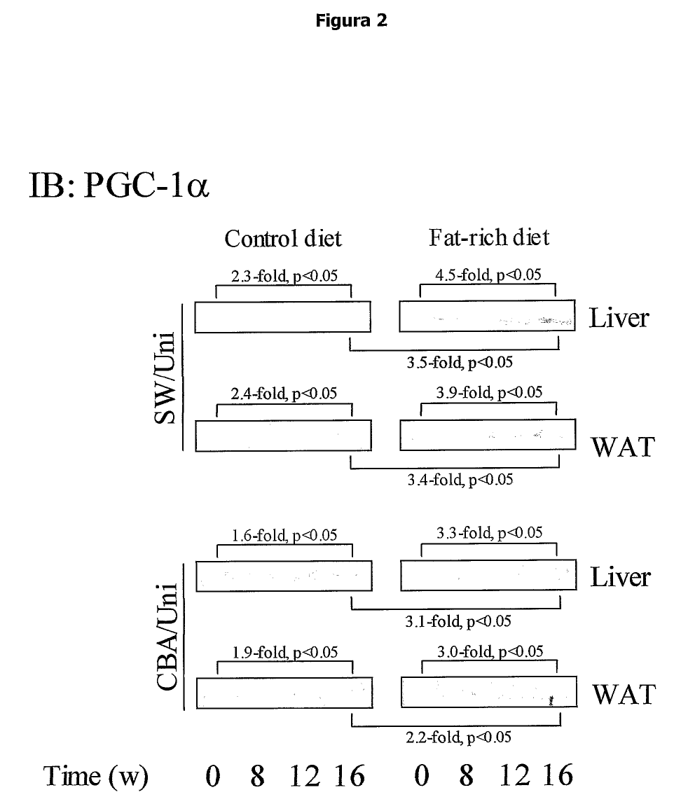

Next, the effect of the lipid-rich diet on the PGC-1α expression in liver and adipose tissue of both strains was determined. For such characterization, fragments of both tissues were obtained from mice of different ages and exposed for variable periods to the standard and lipid-rich diets. Protein extracts obtained from these fragments were used in immunoblot experiments with specific anti-PGC-1α antibodies. The bands obtained on the blots were quantified by digital densitometry and compared to each other. As presented in FIG. 2, the aging as well as the consumption of lipid-rich diet exerted an effect of significant increase on the PGC-1α expression in both tissues. However, as revealed by the statistical analysis, mice from the SW/Uni strain presented greater increases in the PGC-1α expression than the CBA/Uni mice.

### EXAMPLE 2

### Effect of the Treatment of SW/Uni Mice with Antisense Oligonucleotide PGC-1α

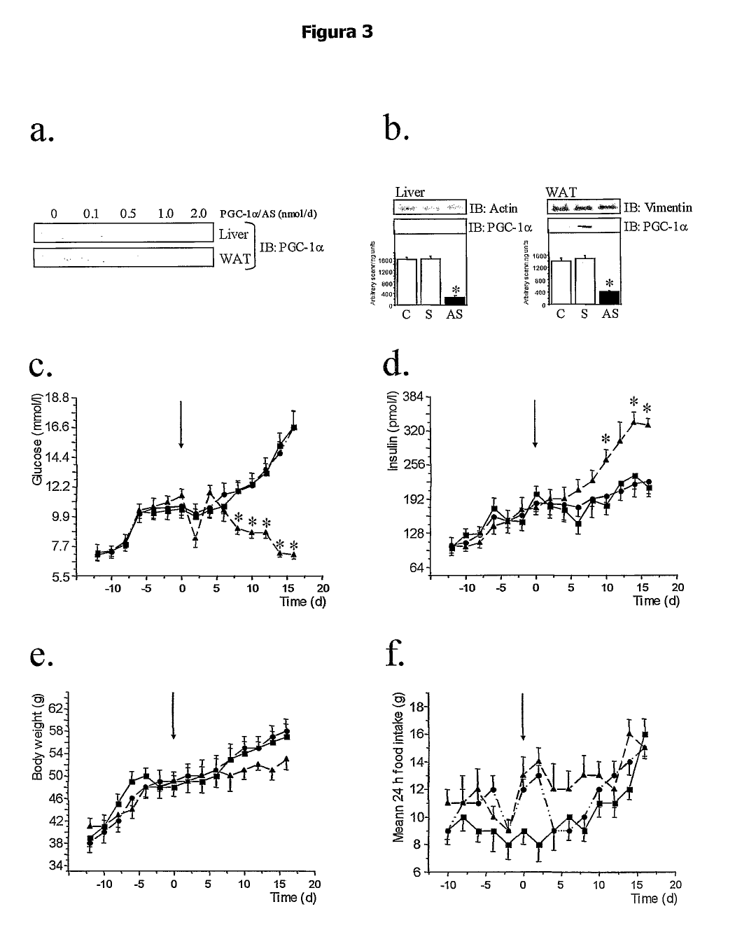

The mice from the SW/Uni strain that developed simultaneously obesity and diabetes mellitus phenotypes when fed with lipid-rich diet were chosen to be the animal model for the tests. In the first part of the characterization the effects of the antisense oligonucleotide PGC-1α (PGC-1α/AS) the immunoblot technique was used in order to evaluate the potency of the compound to inhibit the target protein expression in liver and adipose tissue of the experimental animals. FIG. 3a shows that PGC-1α/AS, when used parenterally for 4 days in SW/Uni mice fed with lipid-rich diet exerts a dose-dependent effect on the target protein expression in liver and adipose. Such effect is specific and does not interfere with the expression of structural proteins (actine and vimentine) of the same tissues (FIG. 3b).

Afterwards, the inhibitory effect of the PGC-1α expression with a single daily dose of 1.0 nmol of PGC-1α/AS on metabolic and hormonal parameters of SW/Uni mice fed with lipid-rich diet. As presented in FIG. 3 (c-f), the compound promoted complete restoration of the baseline serum glucose levels after 16 days of treatment. Such effect was accompanied by a significant increase of the baseline plasmatic insulin levels. Still, there was a tendency of body mass reduction without alteration of food intake.

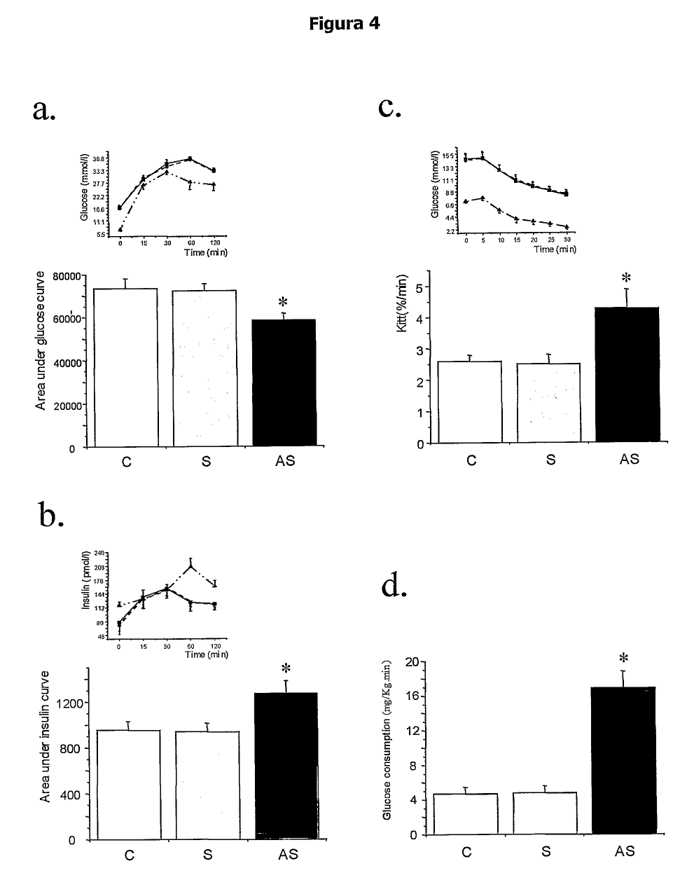

In order to evaluate the effect of the compound on insulin secretion and action in vivo, the SW/Uni mice fed with lipid-rich diet were treated with PGC-1α/AS (1.0 nmol/day), with sense control oligonucleotide or with vehicle and evaluated by the glucose tolerance and insulin tolerance tests and by the euglycemic-hyperinsulinemic clamp. As presented in FIG. 4, the treatment with PGC-1α/AS promoted reduction of the glucose levels and increase of the insulin levels during the glucose tolerance test (FIGS. 4a and b), increase of the glucose decay rate during the insulin tolerance test (FIG. 4c) and increase of the glucose consumption rate during the euglycemic-hyperinsulinemic clamp (FIG. 4d).

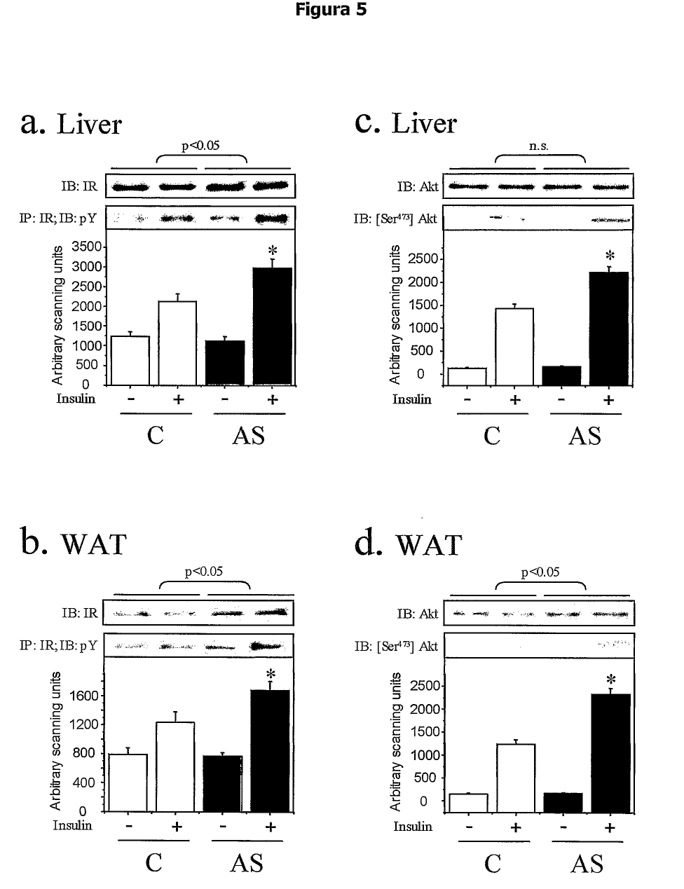

Finally, the effects of the treatment with PGC-1α/AS on the molecular expression and activation of two proteins with important role in insulin action, the insulin receptor (IR) and the Akt signal transducer protein were evaluated. For such, the SW/Uni mice were treated with PGC-1α/AS or control sense oligonucleotide or vehicle, and fragments obtained from liver and adipose tissue were employed in immunoblot and immunoprecipitation experiments and for IR and Akt study. As presented in FIG. 5, the treatment with PGC-1α/AS promoted increase of the IR expression in liver and adipose tissue, and increase of the Akt expression in adipose tissue. The treatment resulted still in increase of the insulin induced IR tyrosine phosphorylation in both tissues and increase of the insulin induced Akt serine phosphorylation in both tissues. In this manner, the inhibition of the PGC-1α obtained through the treatment with PGC-1α/AS exerts important effects on molecular mechanisms of insulin action, favoring the activity of this hormone in target tissues.

The above description of the present invention was presented for the purpose of illustration and description. Besides this, the description does not intend to limit the invention to the form revealed herein. As consequence, variations and modifications compatible with the above instructions and the ability or knowledge of the relevant technique, are within the scope of the present invention.

The modalities described above intend to explain better the known ways for the practice of the invention and to permit the technical experts in the field to use the invention in such, or other, modalities and with several modifications necessary for the specific applications or uses of the present invention. It is the intention that the present invention includes all its modifications and variations and in the attached claims.

1) An OLIGONUCLEOTIDE consisting of 80 synthetic or natural bases corresponding to the following modified or unmodified sequences:
i. Sequence no. 1 (SEQ ID NO:1), 5′-tggagttgaa aaagcttgac tggcgtcatt caggagctgg atggcgtggg acatgtgcaa ccaggactct gagtctgtat-3′;
ii. Sequence no. 2 (SEQ ID NO:2), 5′-tgctctgtgt cactgtggat tggagttgaa aaagcttgac tggcgtcatt caggagctgg atggcgtggg acatgtgcaa-3′;
iii. Sequence no. 3 (SEQ ID NO:3), 5′-tggcgtcatt caggagctgg atggcgtggg acatgtgcaa ccaggactct gagtctgtat ggagtgacat cgagtgtgct-3′; and
iv. a fragment of Sequence no. 1 (SEQ ID NO:1), Sequence no. 2 (SEQ ID NO:2), or Sequence no. 3 (SEQ ID NO:3), that has at least 5 bases.

2) The OLIGONUCLEOTIDE, according to claim 1, wherein the sequence includes the bases of any of the Sequence no. 1 (SEQ ID NO:1), Sequence no. 2 (SEQ ID NO:2), and Sequence no. 3 (SEQ ID NO:3) in the positions from the group consisting of from 1 to 20, from 2 to 21, from 3 to 22, from 4 to 23, from 5 to 24, from 6 to 25, from 7 to 26, from 8 to 27, from 9 to 28, from 10 to 29, from 11 to 30, from 12 to 31, from 13 to 32, from 14 to 33, from 15 to 34, from 16 to 35, from 17 to 36, from 18 to 37, from 19 to 38, from 20 to 39, from 21 to 40, from 22 to 41, from 23 to 42, from 24 to 43, from 25 to 44, from 26 to 45, from 27 to 46, from 28 to 47, from 29 to 48, from 30 to 49, from 31 to 50, from 32 to 51, from 33 to 52, from 34 to 53, from 35 to 54, from 36 to 55, from 37 to 56, from 38 to 57, from 39 to 58, from 40 to 59, from 41 to 60, from 42 to 61, from 43 to 62, from 44 to 63, from 45 to 64, from 46 to 65, from 47 to 66, from 48 to 67, from 49 to 68, from 50 to 69, from 51 to 70, from 52 to 71, from 53 to 72, from 54 to 73, from 55 to 74, from 56 to 75, from 57 to 76, from 58 to 77, from 59 to 78, from 60 to 79, and from 61 to 80.

3). (canceled)

4). (canceled)

5). (canceled)

6). (canceled)

7). (canceled)

8). (canceled)

9). (canceled)

10). (canceled)

11). (canceled)

12). (canceled)

13). (canceled)

14). (canceled)

15). (canceled)

16). (canceled)

17). (canceled)

18). (canceled)

19). (canceled)

20). (canceled)

21). (canceled)

22). (canceled)

23). (canceled)

24). (canceled)

25). (canceled)

26). (canceled)

27). (canceled)

28). (canceled)

29). (canceled)

30). (canceled)

31). (canceled)

32). (canceled)

33). (canceled)

34). (canceled)

35). (canceled)

36). (canceled)

37). (canceled)

38). (canceled)

39). (canceled)

40). (canceled

41). (canceled)

42). (canceled)

43). (canceled)

44). (canceled)

45). (canceled)

46). (canceled)

47). (canceled)

48). (canceled)

49). (canceled)

50). (canceled)

51). (canceled)

52). (canceled)

53). (canceled)

54). (canceled)

55). (canceled)

56). (canceled)

57). (canceled)

58). (canceled)

59). (canceled)

60). (canceled)

61). (canceled)

62). (canceled)

63) The OLIGONUCLEOTIDE, according to claim 1, wherein the sequence includes the bases of any of the Sequence no. 1 (SEQ ID NO:1), Sequence no. 2 (SEQ ID NO:2), or Sequence no. 3 (SEQ ID NO:3) in the position from the group consisting of from 26 to 41, from 27 to 42, from 28 to 43, from 29 to 44, from 30 to 45, from 31 to 46, and from 32 to 47.

64). (canceled)

65). (canceled)

66). (canceled)

67). (canceled)

68). (canceled)

69). (canceled)

70) The OLIGONUCLEOTIDE according to claim 1, further comprising at least one fragment varying between 5 to 79 bases that is contained in the Sequence no. 1 (SEQ ID NO:1), Sequence no. 2 (SEQ ID NO:2), or Sequence no. 3 (SEQ ID NO:3).

71) A PHARMACEUTICAL COMPOUND for the manufacture of a medication wherein the compound comprises an oligonucleotide according to claims 1, 2, 63 or 70.

72) The PHARMACEUTICAL COMPOUND, according to claim 71, wherein the medication administration route is enteral.

73) A method of treating diabetes mellitus, insulin resistance, and/or metabolic syndrome comprising the step of administering to an individual with diabetes mellitus, insulin resistance, and/or metabolic syndrome THE PHARMACEUTICAL COMPOUND of claim 71.

74) The PHARMACEUTICAL COMPOUND of claim 71 in a pharmaceutically effective quantity, having a pharmaceutically effective quantity of the oligonucleotide, and further comprising at least one of a pharmaceutically effective quantity of vehicles, diluents, solvents and excipients, pharmaceutically acceptable for therapeutic application.

75) The PHARMACEUTICAL COMPOUND according to claim 71 or 74
wherein the medication is for the treatment of diabetes mellitus, insulin resistance and metabolic syndrome.

76) The PHARMACEUTICAL COMPOUND according to claim 74 wherein the pharmaceutically effective quantity of the compound is from about 200 nMol to about 2000 nMol per dose.

77). (canceled)

78) A method of treating diabetes mellitus comprising the step of administering to a individual with diabetes mellitus THE PHARMACEUTICAL COMPOUND of claim 74.

79) An EXPRESSION VECTOR for the manufacture of medications for the treatment of diabetes mellitus, insulin resistance and metabolic syndrome wherein the vector comprises a sequence corresponding to the oligonucleotide of claims 1, 2, 63, or 70 and wherein the vector is capable of transforming a host cell in a bioreactor of the compound of claims 71 or 72.

80) The pharmaceutical compound of claim 71 wherein the oligonucleotide is modified.

81) The pharmaceutical compound of claim 71 wherein the oligonucleotide is unmodified.

82) The pharmaceutical compound of claim 71 wherein the oligonucleotide is synthetic.

83) The pharmaceutical compound of claim 71 wherein the oligonucleotide is natural.

84) The pharmaceutical compound of claim 71 wherein the medication administration route is parenteral.

85) A method of treating insulin resistance comprising the step of administering to an individual with insulin resistance the pharmaceutical compound of claim 74.

86) A method of treating metabolic syndrome comprising the step of administering to an individual with metabolic syndrome the pharmaceutical compound of claim 74.
